(19) United States
(12) Patent Application Publication
(10) Pub. No.: US 2007/0112041 A1
(43) Pub. Date: May 17, 2007

(54) Nicardipine injection composition

(75) Inventors: Subhas Balaram Bhowmick, Baroda (IN); Ajay J. Khopade, Baroda (IN); Sharmila Ganesh, Baroda (IN)

Correspondence Address: SUGHRUE MION, PLLC, 2100 PENNSYLVANIA AVENUE, N.W., SUITE 800, WASHINGTON, DC, 20037, US

(73) Assignee: SUN PHARMACEUTICAL INDUSTRIES LIMITED

(21) Appl. No.: 11/598,746

(22) Filed: Nov. 14, 2006

(30) Foreign Application Priority Data
Nov. 16, 2005 (IN) 1428/MUM/2005

(51) Int. Cl. A61K 31/455

(52) U.S. Cl. 514/355

(57) ABSTRACT

A nicardipine hydrochloride injection composition comprising sorbitol in amounts sufficient to stabilize the injection preparation and a physiologically and pharmaceutically acceptable buffer, in an amount effective to maintain the pH of the composition at about 3.0 to about 4.5.

The present invention relates to an injection composition of nicardipine.

### BACKGROUND OF THE INVENTION

Nicardipine hydrochloride is a dihydropyridine derivative with IUPAC (International Union of Pure and Applied Chemistry) chemical name (±)-2-(benzyl-methyl amino) ethyl methyl 1,4-dihydro-2,6-dimethyl-4-(m-nitrophenyl)-3,5-pyridinedicarboxylate monohydrochloride. Nicardipine hydrochloride is a calcium ion influx inhibitor (slow channel blocker or calcium channel blocker) and is indicated for the short-term treatment of hypertension when oral therapy is not feasible or not desirable.

Nicardipine hydrochloride is available in the US as CARDENE I.V. for intravenous administration, CARDENE capsules and CARDENE SR extended release capsules for oral administration. CARDENE I.V. (ESP Pharma) for intravenous administration contains 2.5 mg/mL of nicardipine hydrochloride and is available as a sterile, non-pyrogenic, clear, yellow solution in 10 ml ampoules for intravenous infusion after dilution, each 1 ml of the concentrate containing 2.5 mg nicardipine hydrochloride in Water for Injection, USP with 48.00 mg sorbitol, NF, buffered to pH 3.5 with 0.525 mg citric acid monohydrate, USP and 0.09 mg sodium hydroxide, NF. Additional citric acid and/or sodium hydroxide may have been added to adjust pH.

U.S. Pat. No. 4,880,823 (the '823 patent) relates to a stable, injectable composition of nicardipine hydrochloride comprising an aqueous nicardipine hydrochloride solution containing 0.04 to 0.6 W/V % nicardipine hydrochloride and 2 to 7 W/V % of a polyhydric alcohol and wherein the pH of said solution is from 2.5 to 5. The patent teaches that the problem of stability of nicardipine hydrochloride formulations associated with chloride containing isotonising agents was resolved by dissolving nicardipine chloride in water together with 2 to 7 W/V % of a polyhydric alcohol and adjusting the pH of the solution to 2.5 to 5. The pH of the solution was controlled by a mineral acid such as hydrochloric acid and/or by a base such as sodium hydroxide. Though the parenteral solution of the formulation, taught by this patent was stable, it was found that upon autoclaving, the pH of the solution would change, leading to precipitation of the nicardipine.

U.S. Pat. No. 5,164,405 (the '405 patent) relates to pharmaceutical composition suitable for parenteral administration comprising (a) a physiologically and pharmaceutically acceptable non-chloride compound selected from saccharides, including sorbitol, mannitol, dextrose and glucose, and non-saccharides, including polyethylene glycol and glycerol, in an amount effective to render the pharmaceutical composition isotonic; (b) a physiologically and pharmaceutically acceptable buffer, selected from citrate, acetate, phosphate, and lactate buffers, in an amount effective to maintain the pH of the composition at about 3.0 to about 4.5; (c) a pharmaceutically acceptable aqueous vehicle consisting essentially of water; and (d) at least about 1 mg/ml nicardipine hydrochloride in solution. The patent specifically discloses use of about 48-50 mg/ml of sorbitol as the preferred non-chloride compound to make the composition isotonic. This patent overcomes the problems associated with compositions of the '823 patent in that the buffered composition of the '405 patent was stable upon terminal sterilization such as autoclaving.

It can be seen that the prior art has achieved stable injection formulations of nicardipine hydrochloride using sorbitol as the isotonicity adjuster. The '405 patent as well as the CARDENE I.V. formulation uses 48-50 mg/ml of sorbitol i.e. 4.8-5.0 W/V % of sorbitol in order to make stable isotonic injection compositions of nicardipine hydrochloride.

It is a well-known fact that parenteral compositions should contain bare minimum excipients to avoid any possible side effects associated with the excipients and associated impurities in the excipient, since the compositions are introduced directly into the systemic circulation.

### OBJECT OF THE INVENTION

It is an object of the present invention to provide nicardipine hydrochloride injection compositions comprising lower amounts of sorbitol. It is a further object of the present invention to provide nicardipine hydrochloride injection compositions comprising sorbitol in lower amounts without compromising on stability and safety of the composition.

### SUMMARY OF THE INVENTION

The present invention provides a nicardipine hydrochloride injection composition comprising sorbitol in amounts sufficient to stabilize the injection preparation and a physiologically and pharmaceutically acceptable buffer, in an amount effective to maintain the pH of the composition at about 3.0 to about 4.5.

In one embodiment, the present invention provides a nicardipine hydrochloride injection composition comprising a single dose comprising:

(a) about 2.5 mg of Nicardipine hydrochloride;

(b) about 20 mg of sorbitol;

(c) a physiologically and pharmaceutically acceptable buffer, in an amount effective to maintain the pH of the composition at about 3.0 to about 4.5;

(d) a pharmaceutically acceptable aqueous vehicle.

### DESCRIPTION OF THE INVENTION

The present invention relates to a nicarclipine hydrochloride injection composition comprising sorbitol in amounts sufficient to stabilize the injection preparation and a physiologically and pharmaceutically acceptable buffer, in an amount effective to maintain the pH of the composition at about 3.0 to about 4.5.

More particularly, the present invention relates to a nicardipine hydrochloride injection composition comprising a single dose comprising:

(a) about 2.5 mg of Nicardipine hydrochloride;

(b) about 20 mg of sorbitol;

(c) a physiologically and pharmaceutically acceptable buffer, in an amount effective to maintain the pH of the composition at about 3.0 to about 4.5;

(d) a pharmaceutically acceptable aqueous vehicle.

The composition of the present invention uses sorbitol in amounts sufficient to act as a cosolvent for the nicardipine hydrochloride so as to prevent its precipitation and maintain stability of the composition. But the amount of sorbitol used is not sufficient to make the composition isotonic. The tonicity of the nicardipine hydrochloride composition is very low, but upon dilution with 0.9% sodium chloride or 5% dextrose, the composition becomes isotonic, so that the final composition which is administered to the patient does not cause irritation.

The composition of the present invention uses nicardipine or its pharmaceutically acceptable salts. The preferred salt used in the composition of the present invention is nicardipine hydrochloride. Nicardipine hydrochloride is used in a “therapeutically effective amount.” By “therapeutically effective amount” as applied to nicardipine hydrochloride is meant that amount which when administered to mammals, especially human patients, will have a calcium entry blocking effect that will be useful to treat their disease conditions, that is, conditions which may be alleviated by the administration of calcium channel blocking agents, especially cardiovascular and cerebrovascular disease conditions. It is preferred that that the nicardipine hydrochloride is used in amounts ranging from about 0.5 mg to about 6 mg for a single dose injection. The more preferred embodiments use about 2.5 mg of nicardipine hydrochloride per single dose injection.

The composition of the present invention uses a polyhydric compound as a cosolvent for preventing precipitation of the nicardipine hydrochloride. The amount of the polyhydric compound used in the composition is such that it is sufficient to prevent the precipitation of the nicardipine hydrochloride but is insufficient to make the composition isotonic. The polyhydric compounds used may be selected from saccharides and non-saccharide polyhydric compounds. Suitable saccharides include sorbitol, mannitol, dextrose and glucose. Suitable polyhydric compounds which are non-sugar compounds include polyethylene glycol (PEG) and glycerol. The preferred polyhydric compound used in the composition of the present invention is sorbitol. It is preferred that sorbitol is used in amounts ranging from about 20 mg to about 35 mg per single dose injection. In a most preferred embodiment, about 20 mg of sorbitol is used per single dose on the injection.

The pharmaceutically acceptable buffer may be selected from any of the buffers that are effective to maintain the pH in the range of about 3.0-4.5, which buffers are well known in the art to which this invention relates. More preferably, the buffer may be selected from citrate, acetate, phosphate and lactate buffers. Most preferably, the buffer is a citrate or acetate buffer, for example, citric acid plus sodium hydroxide in appropriate proportions which will maintain the pH at about 3.5-4.5. Use of citrate buffer in an amount sufficient to maintain a concentration in the range of 0.002M to 0.003M has been found to afford advantages in maintaining the pH of the composition in the range of about 3.5-4.5. The buffer system is useful over the desired dose range of the composition to provide ease of manufacture of the composition, to maintain pH stability during and after manufacture including terminal sterilization by autoclaves and thus to render the composition compatible with a range of infusion fluids.

The pharmaceutically acceptable carrier in the pharmaceutical composition may be selected from 100% of water (water for injection) or an aqueous system (that is an aqueous vehicle) comprising at least a major proportion of water.

The injection composition of the present invention may be available as a concentrate composition to be mixed with infusion fluids such as 0.9% sodium chloride or 5% dextrose before administration to the patient, or ready to use compositions which are premixed with infusion fluids such as 0.9% sodium chloride or 5% dextrose.

The examples that follow do not limit the scope of the invention and are merely used as illustrations.

### EXAMPLE 1

The pharmaceutical composition of the present invention was obtained as per the method given in Table 1 below.

TABLE 1

| Sr. No. | Ingredients | mg/ml | % W/V |
|---|---|---|---|
| 1. | Nicardipine hydrochloride | 2.5 | 0.25 |
| 2. | Sorbitol | 20 | 2.0 |
| 3. | Citric acid monohydrate | 0.525 | 0.0525 |
| 4. | Sodium hydroxide | 0.09 | 0.009 |
| 5. | Water For Injection | to 1 ml | |

pH of the composition adjusted to 3.5-4.0

The citric acid was first dissolved in 80% V/V of the Water For Injection (WFI) of the batch size. Sorbitol was then added to the above solution and stirred till it dissolved. Sodium hydroxide was then dissolved in the above solution. The nicardipine hydrochloride was dissolved in the above solution. The volume of the composition was then made up with the WFI and the pH adjusted to 3.5-4.0, if required.

### EXAMPLE 2

Tonicity studies were carried out on the injection composition of example 1 and compared with the tonicity of the marketed injection composition CARDENE I.V. The tonicity of the injection composition, concentrate and solutions obtained on dilution of 1 part of the concentrate injection composition in 24 parts of 0.9% sodium chloride or dextrose 5%, are recorded in Table 2 below.

TABLE 2

| | | Tonicity | |
|---|---|---|---|
| Composition and its | | | Composition of |
| reconstitution | | CARDENE I.V. | example 1 |
| 1 | Nicardipine injection | 282 | 110 |
| 2 | Nicardipine injection | 266 | 262 |
| | (1 part) diluted with | | |
| | 0.9% sodium chloride | | |
| | (24 parts) | | |
| 3 | Nicardipine injection | 267 | 256 |
| | (1 part) diluted with | | |
| | dextrose 5% (24 parts) | | |

It is evident that although the concentrate obtained in the present invention has lower tonicity as compared to CARDENE I.V., the diluted preparations (which are administered to patients) have comparable tonicity values.

Other modifications and variations to the invention will be apparent to those skilled in the art from the foregoing disclosure and teachings. Thus, while only certain embodiments of the invention have been specifically described herein, it will be apparent that numerous modifications may be made thereto without departing from the spirit and scope of the invention

What is claimed is:

1. A nicardipine hydrochloride injection composition comprising sorbitol in amounts sufficient to stabilize the injection preparation and a physiologically and pharmaceutically acceptable buffer, in an amount effective to maintain the pH of the composition at about 3.0 to about 4.5.

2. A nicardipine hydrochloride injection composition comprising a single dose comprising:
(a) about 2.5 mg of Nicardipine hydrochloride;
(b) about 20 mg of sorbitol;
(c) a physiologically and pharmaceutically acceptable buffer, in an amount effective to maintain the pH of the composition at about 3.0 to about 4.5;
(d) a pharmaceutically acceptable aqueous vehicle.
